(19) United States
(12) Patent Application Publication
(10) Pub. No.: US 2022/0054820 A1
(43) Pub. Date: Feb. 24, 2022

(54) WEARABLE DEVICES

(71) Applicant: Carl Turner, Eugene, OR (US)

(72) Inventor: Carl Turner, Eugene, OR (US)

(73) Assignee: Carl Turner, Eugene, OR (US)

(21) Appl. No.: 17/390,210

(22) Filed: Jul. 30, 2021

Related U.S. Application Data
(60) Provisional application No. 63/059,085, filed on Jul. 30, 2020.

(51) Int. Cl. A61N 1/04; A61N 1/08; A61N 1/30

(52) U.S. Cl. CPC A61N 1/0484; A61N 1/0444; A61N 1/08; A61N 1/325; A61N 1/0452; A61N 1/0456; A61N 1/30

(57) ABSTRACT

This disclosure provides self-applied wearable devices configured to electrically stimulate a user, generate and/or collect a user's electrophysiological data via electrodes, in contact with the outer layer of skin, that hydrate the skin surface using iontophoresis, reverse iontophoresis, and/or a combination thereof. The wearable devices provided herein increase conductivity and/or move biological ions and/or polar molecules from the electrode into the outer skin layer of skin surface to reduced impedance between the outer skin layer and electrode, wherein the impedance between electrodes is matched and/or minimized. This disclosure also provides systems comprising the same, and methods for making and using the same.

### RELATED APPLICATIONS

This application claims priority to provisional application Nos. U.S. Ser. No. 63/059,085 filed Jul. 30, 2020, which is hereby incorporated into this application in its entirety.

### FIELD OF THE DISCLOSURE

This disclosure relates to self-applied wearable device(s) configured to electrically stimulate a user, generate and/or collect a user's electrophysiological data using iontophoresis, reverse iontophoresis and/or a combination thereof to hydrate the outer layer of skin in contact with one or more electrodes.

### BACKGROUND OF THE DISCLOSURE

In the field of health and wellness, wearable technology makes use of electrophysiology to collect data about a person's health. Non-invasive electrophysiology is used by clinicians and researchers to study the health and wellness of humans. Common measurements include electrocardiography (ECG), electromyography (EMG), electrooculography (EOG) and electroencephalography (EEG). These measurements can be used for a variety of medical diagnostic, research purposes or health and wellness monitoring across a wide variety of fields including neurological, physical sciences, cognitive neuroscience, cardio health, general health, sleep etc. The quality of data generated by the acquisition system is fundamentally and critically important to reaching the correct medical diagnosis, scientific conclusions or providing the correct recommendations. The techniques used in extracellular electrophysiology involve collecting an electrical signal from a human or animal body. There are also techniques for intracellular ion channel recording and for sub-dermal and invasive monitoring. To measure electrical activity, electrodes are used to pick up the electrical signal from the skin. Electrodes bring electrical potential to an instrumentation amplifier to amplify a weak electrical signal to something that can be observed by either analog electronics or digitized by an analog to digital converter (ADS). Electrodes can pick up signals in one of two ways, either through capacitive coupling or direct electrical contact. Capacitive electrodes are susceptible to environment noise and movement artifacts. This makes capacitive electrodes notoriously noisy to use in most practical situations especially for tiny signals like EEG.

For contact electrodes, the magnitude and stability of the electrode—skin impedance largely affect the quality of electrical recordings. (“Bio-Integrated Wearable Systems: A Comprehensive Review . . . ” Jan. 28, 2019, https://pubs.acs.org/doi/10.1021/acs.chemrev.8b00573. Accessed Jul. 11, 2020). To achieve high quality recordings, it is important that impedance levels of the electrode to skin interface remain low relative to the input impedance of front-end amplifiers. In addition, matched impedance between electrodes is important for common mode rejection. Often clinical procedures will dictate a threshold of electrode impedance required before collecting a recording. For example the American Academy of Sleep Medicine (AASM) scoring manual (“AASM Scoring Manual—American Academy of Sleep Medicine.” Mar. 4, 2020, https://aasm.org/clinical-resources/scoring-manual/. Accessed Jul. 11, 2020) recommends electrodes used for EMG to be less than 10 kOhms but preferred to be less than 5 kOhms. For EEG impedances are recommended to be less than 5 kOhms.

The main factor contributing to high impedances is the skin and skin to electrode interface. Specifically, the outermost layer of the epidermis, the stratum corneum, is a barrier to electrical current. Clinical practice often requires abrading the skin to remove this outer layer. This can be irritating to the skin and increase the risk of infection. Another common clinical practice is to use electrolyte (e.g. Ten20 conductive paste)—a conductive solution to penetrate the skin and make physical and electrical contact between the skin and electrode. Pastes are sticky and messy, hard to clean, get stuck in hair—generally a pain for a patient. Iontophoresis is known as a technology used to move molecules through skin using electrical current, where typically the item being transported is medication. The same technology can be used to move naturally occurring conductive molecules, or polar molecules such as H₂O to hydrate, and lower impedance of the skin (“Use of electroporation and reverse iontophoresis for extraction . . . ” Feb. 22, 2012, https://www.ncbi.nlm.nih.gov/pmc/articles/PMC3289445/. Accessed Jul. 11, 2020; “Direct current conditioning to reduce the electrical impedance . . . ” Nov. 8, 2017, https://www.researchgate.net/publication/320867259_Direct_current_conditioning_to_reduce_the_electrical_impedance_of_the_electrode_to_skin_contact_in_physiological_recording_and_stimulation. Accessed Jul. 11, 2020). Typical electrodes used in clinical and research settings and sometimes with wearable devices are hard plastics or other hard polymers. Those are uncomfortable for the user especially over time. Atopic contact dermatitis can be caused by electrodes that are pressed against the skin. If using hard electrodes without gel or paste, the irritation can be substantially worse for the subject. In addition, typical systems for measuring clinical quality EEG and current injection systems are large cart-based systems and require a trained technician to connect to a user.

This disclosure provides solutions to these and other problems. For instance, in some embodiments, this disclosure provides devices and methods thereof for using electrodes that have direct contact with skin. In some embodiments, the devices disclosed herein have the capability to both lower impedance and match impedances. In some embodiments, the devices disclosed herein use soft, biocompatible conductive polymer electrodes, optionally with hydration that can pull conductive related molecules from deeper layers of the skin to the outermost layers by iontophoresis to reduce impedance and/or push conductive-related molecules from the electrode into the outermost layer of the skin. And in some embodiments, this disclosure provides devices that include a built-in automatic signal quality detection and impedance adjustment optimization algorithm. In some embodiments, the devices disclosed herein do not require clinicians and other attendants to be present to prepare, measure, monitor and adjust electrodes to ensure adequate signal quality. In addition, the devices disclosed herein provide easy-to-use wearable devices to be worn in everyday life, such as during sleep and/or exercise. This disclosure thereby provides solutions to these and other art-recognized, and unrecognized, problems.

### SUMMARY OF THE DISCLOSURE

This disclosure relates to a self-applied wearable device(s) configured to electrically stimulate a user, generate and/or collect a user's electrophysiological data, comprising one or more electrodes wherein said one or more electrodes are in contact with an outer layer of a skin surface of the user and hydrate the outer layer of the skin via iontophoresis, reverse iontophoresis, and/or a combination thereof to move biological ions and/or polar molecules from beneath the skin surface into the outer skin surface to increase conductivity and/or to move the molecules from the electrode into the outer skin layer of skin surface to reduced impedance between the outer skin layer and electrode, and wherein the impedance between electrodes is matched and/or minimized. In some embodiments, this disclosure relates to systems comprising and methods for using the same. Other embodiments are also contemplated herein as would be understood by those of ordinary skill in the art.

### BRIEF DESCRIPTION OF THE DRAWINGS

FIG. 1 illustrates exemplary wearable devices.

FIG. 2 provides a block diagram showing the major components of an embodiment disclosed herein.

FIG. 3 illustrates the migration of molecules between skin and electrode during iontophoresis and reverse iontophoresis which can optimize impedance.

### DETAILED DESCRIPTION OF THE INVENTION

This disclosure relates to systems, devices, and methods that use electrodes with iontophoresis, reverse iontophoresis and/or a combination thereof to optimize signal quality from wearable devices by hydrating the outer layer of skin, which avoids the use of conductive solutions or abrasion of the skin, wherein the impedance between electrodes is matched and/or minimized. As used herein, matched an/or minimized impedance between electrodes means a difference of less than 30 kOhms between electrodes, less than 20 kOhms, less than 15 kOhms, less than 10 kOhms, or less than 5 kOhms between electrodes. In certain embodiments, matched an/or minimized impedance between electrodes means a difference of about 25 kOhms to about 5 kOhms, or a difference of about 20 kOhms to about 10 kOhms between electrodes. In embodiments, the devices (and systems thereof) are self-applied wearable devices configured to electrically stimulate a user, generate and/or collect a user's electrophysiological data. As used herein “iontophoresis” refers to a process wherein a small electrical current is used to move (e.g. enhance transport) of charged biological ions (e.g. sodium or potassium) and/or polar molecules (e.g. water, H₂O) from the electrode into or through the outer layer of the skin surface. In this way, iontophoresis hydrates the skin surface providing conductive ions or polar molecules from the electrode (e.g. hydrogel electrodes). In certain embodiments, iontophoresis may further be used for transdermal drug delivery by use of a voltage gradient on the skin. In other words, molecules are transported across the stratum corneum by electrophoresis and electroosmosis. In certain embodiments, iontophoresis can also increase the permeability of the skin. As used herein “reverse iontophoresis” refers to a process using a small electrical current to move or extract molecules (e.g., biological ions and/or polar molecules) from beneath the skin surface (within the body) to the skin surface. In this way, reverse iontophoresis hydrates the skin surface providing conductive ions or polar molecules such as H₂O from beneath the skin surface. The negative charge of the skin at buffered pH causes it to be permselective to cations such as sodium and potassium ions, allowing iontophoresis which causes electroosmosis, solvent flow towards the anode. In embodiments, the use of electrodes for iontophoresis, reverse iontophoresis and/or a combination thereof hydrates the outer layer of skin when the electrodes are in contract with the skin.

In embodiments, the present self-applied wearable devices comprise one or more dry electrodes. As used herein “dry electrodes” refer to an electrode configured for iontophoresis and/or reverse iontophoresis. In embodiments, the dry electrodes comprise a flexible, compliant, conductive polymer such as a hydrogel or silicone based polymer. In certain embodiments, the dry electrodes herein are not selected from metal “disc” electrodes, such as those made from stainless steel, tin, gold or silver covered with a silver chloride coating, or those electrodes that comprise a hard polymer plastic. In embodiments, the optimization of impedance from hydration of the outer layer of skin is accomplished using a combination of conductive polymers, materials, electronics, hardware, software and/or algorithms, as disclosed herein or as may additionally be suitable for use and available to those of ordinary skill in the art. The system typically comprises a set electrodes, electronics, and wires connecting the electrodes to the electronics. In some embodiments, the electronics can comprise of one or more input conditioning circuit(s), multiplexer(s), instrumentation amplifier(s), analog to digital converter(s), microprocessor(s), radio frequency (RF) hardware, wireless antenna(s), data storage system(s), current source(s)/sink(s), firmware and impedance optimization algorithm(s). Other embodiments are also contemplated herein as would be understood by those of ordinary skill in the art.

In some embodiments, the impedance optimization algorithm is implemented in software to automatically optimize the impedance and thus signal quality and/or electrical connection of the electrodes. In some embodiments, the algorithm can run embedded on the wearable device or on a host computer or smartphone.

In preferred embodiments, the electrodes, wire(s) and electronics can be part of any apparatus in contact with the body for the purpose of recording an extracellular, electrophysiological data recording. For example the device can be in a wearable format, such as but not limited to a head band, head sensor net, cap, earpiece, ring, arm band, watch, leg band or other similar device. The systems, devices and methods of this disclosure can be especially important for wearable devices that are worn in situations in which a clinical attendant is not present to prepare, measure, monitor and adjust electrodes to ensure adequate signal quality. In embodiments, the device is configured to electrically stimulate a user, generate and/or collect a user's electrophysiological data. In some embodiments, the systems, devices and methods can also be used to improve electrode impedance for the purpose of electrically stimulating a user, such as transcranial direct current stimulation (tDCS) or electrical muscle stimulation (EMS) devices. In certain embodiments, electrophysiological data comprises and/or selected from electroencephalogram (EEG), electrooculography (EOG), electrocardiogram (ECG), electroatriography (EAG), electroventriculography (EVG), intracardiac electrogram (EGM), electrocorticography (ECoG or iEEG), electromyography (EMG), electroretinography (ERG), electronystagmography (ENG), electroolfactography (EOG), electrocochleography (ECOG or ECochG), electrogastrography (EGG), electrogastroenterography (EGEG), and electromyography (EMG).

In some embodiments, the electrodes included in the devices of this disclosure are designed to provide low, matched, and stable impedance while being comfortable and hassle-free for the user. In some embodiments, biocompatible base polymers such as polydimethylsiloxane (PDMS)-based polymers, and/or other commercially available materials, can be used to construct the electrodes. For example, in some embodiments, Dupont MG 7-9900 Soft Skin Adhesive, Dupont MG-2402 Pressure Sensitive Adhesive and/or Sylgard 184 can be used. In some embodiments, conductive particles such as silver particles, silver threads, carbon particles, carbon nanotubes, graphene and Ag/AgAl coating can be included to improve the conductivity of the electrodes (see, e.g., U.S. Pat. Publ. 2019/0214593 A1). In some embodiments, hydrogels can be used for electrodes for cases where applying molecules (H₂O) onto the skin surface from the electrode to hydrate is desirable. In some embodiments, hydrogels composed of cross-linked polymers (see, e.g., U.S. Pat. No. 8,706,183 B2) such as polyAMPS can be included. In some embodiments, electrodes can be coated with an Ag/AgCl mixture to allow current flow with minimal impedance (“Evaluating Major Electrode Types for Idle Biological Signal . . . ” Aug. 24, 2016, https://www.ncbi.nlm.nih.gov/pmc/articles/PMC5597189/. Accessed Jul. 11, 2020). The malleable nature of the PDMS elastomer, for example, physically allows more surface area of the electrode to physically contact the skin which can also improve impedance. The electrodes disclosed in some embodiments of this disclosure are much preferable than hard-disk shaped Ag/AgAl electrodes that are ubiquitous in clinical use. These are also preferred as compared to pins and/or spike arrays as these are less iterating to the skin and more surface area. In some embodiments, the electrodes are dry electrodes, which the skilled artisan would understand means that a conductive solution, such as but not limited to a gel, that reduces impedance between outer skin layer and electrode is not required since the device accomplishes this without such a conduction solution.

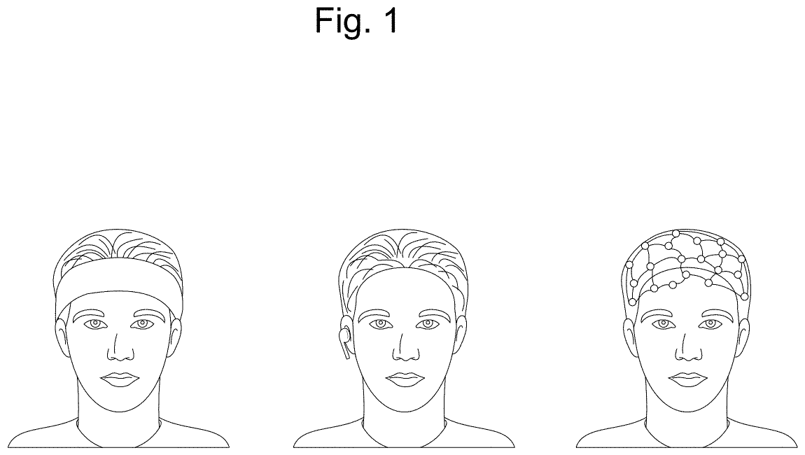

In some embodiments, the system and devices use the latest advances in consumer electronics and materials to provide a comfortable, wearable device. In some embodiments, the device takes the form of a headband, with the electronics enclosure being on the forehead, top of the head, and/or back of the head, depending on the user preferences and depending on the application for where it is used (see FIG. 1). In some embodiments, the electronics can be incorporated into a ring, an earpiece (“[2003.00657] Wireless User-Generic Ear EEG—arXiv.” Mar. 2, 2020, https://arxiv.org/abs/2003.00657. Accessed Jul. 11, 2020)), noise piece, wristband, legband, worn around the neck, chest, and/or any other part of the body. In some embodiments, in order to improve comfort of the device, a small, unobtrusive design that incorporates microchips and integrated circuits, as described in more detail herein. In some embodiments, the electronics can be connected to the electrodes via tangle-free suitable gauge (e.g., 28) light wire(s) (e.g., colored), although any suitable wire may be used. In some embodiments, wires can be connected to the electrodes at time of electrode curing and/or can be heat-staked, sonically-welded and/or connected via conductive epoxy or other suitable material. In some embodiments, disposable electrodes with a button snap interface can be used. In some embodiments, the opposite end of the electrode wire connects to the electronics using an electronic connector. In some embodiments, electrodes that are close to the electronics enclosure can be connected directly to the circuit board using connectors instead of wires, or be part of the rigid or flexible PCB. In some embodiments, and to maintain the position of electrodes in relation to the electronics enclosure, one or more comfortable, biocompatible, stretchable silicone elastomers can be used. In one embodiment, the electrodes can be positioned in a geodesic sensor net (see, e.g., U.S. Pat. No. 5,291,888 A).

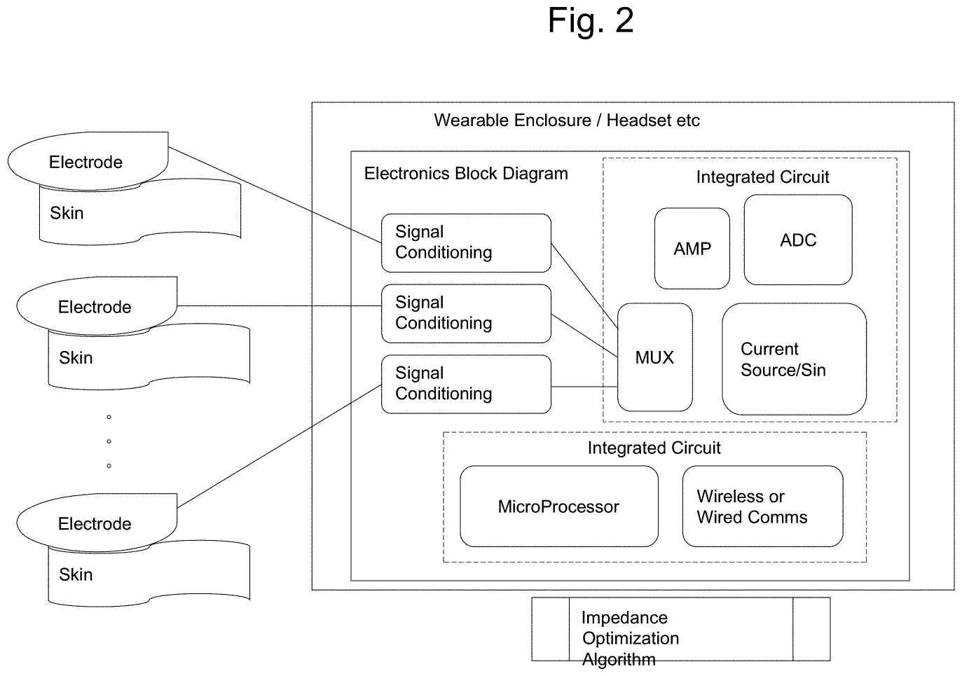

In preferred embodiments, the electronics design of the devices of this disclosure are optimized for wearable devices, with the primary design constraints being miniature size and low power. The elements of one embodiment of the electronics is illustrated in the block diagram provided by FIG. 2. The figure shows electrodes in contact with a user's skin and the electrodes electrically connected to the wearable electronics. The system and devices of this disclosure typically include one or more multiplexers (MUX), which is a series of solid state dynamic interconnects and switches to allow the electrodes to be connected to the signal conditioning, ADC, current source/sink elements as needed. In some embodiments, the multiplexer can be implemented using discrete solid state switches, a programmable array logic (PAL) device, a complex programmable logic device (CPLD), a field programmable gate array (FPGA), or a combination of these technologies. In preferred embodiments, the MUX(s) can also be included with an integrated circuit, such as but not limited to the TI ADS1299 (see block diagram, FIG. 2).

In some embodiments, the system and devices of this disclosure also typically includes one or more components for signal conditioning (see block diagram, FIG. 2), which is a series of passive components to protect users and electronics from static discharge, faults, and/or filter high-frequency RF noise prior to the analog-to-digital converter (ADC). In some embodiments, this can be implemented using a PI circuit with dual resistors for redundant user protection.

In some embodiments, the system and devices of this disclosure also typically includes one or more Amplifiers (AMP) and Analog to Digital Converters (ADC) (see block diagram, FIG. 2). that convert the analog signal into a digitized signal. Such an AMP or ADC can be one of many commercially available low power, miniature integrated chips that perform both functions such as the ADS1299, or other suitable component.

In embodiments, the system and devices of this disclosure comprise one or more Current Generators (CG) (see block diagram, FIG. 2), which is a set of chips or part of an integrated chip, such as the ADS1299, that has the ability to generate current in different types of waveforms. The CG can be DC, AC sinusoidal, and/or square wave at various, preferably out of band, frequencies, such as DC or 125 Hz. In some embodiments the maximum current amplitude will be in the range of 5 to 50 microamps. There may one be one current generator on the device in which case its path of current will have to be multiplexed to the desired electrodes and only one current path at a time will exist. In embodiments with more that one CG multiple current paths may exist simultaneously.

In some embodiments, the system and devices of this disclosure also typically includes one or more microprocessors and components for communications (see block diagram, FIG. 2). For instance, in some embodiments, the nRF52 can be used as it provides low power, very small size, and Bluetooth Low Energy (BLE) wireless communications. In preferred embodiments, the microprocessor includes or runs on microprocessor firmware that implements part of the impedance optimization algorithm and is controlled by software running on the host (e.g., a smartphone or other computer).

In some embodiments, the system and devices of this disclosure are typically controlled by an Impedance Optimization Algorithm (IAO) (see block diagram, FIG. 2). In preferred embodiments, the IAO: 1) checks the impedance of at least three electrode pairs to deduce the impedance of each individual electrode; 2) uses electrodes with lowest impedance to calculate impedances of the other electrodes; determines electrodes that are not connected, lead off detection; 3) determines electrodes that are bridged or have a short circuit with other electrodes; and, 4) applies current according to pre configured settings, to connected, non-bridged electrodes (highest impedance electrodes as sources, sinks with lowest impedance, and vice versa depending on settings and according to rotation algorithm). In preferred embodiments, step 1 can be repeated until within desirable tolerances or best optimization possible during a set time limit. The impedance optimization algorithm rotates electrodes in use and changes the polarity of electrodes used for impedance conditioning to minimize the oxidation that occurs at each electrode, thereby prolonging the life span of the electrodes.

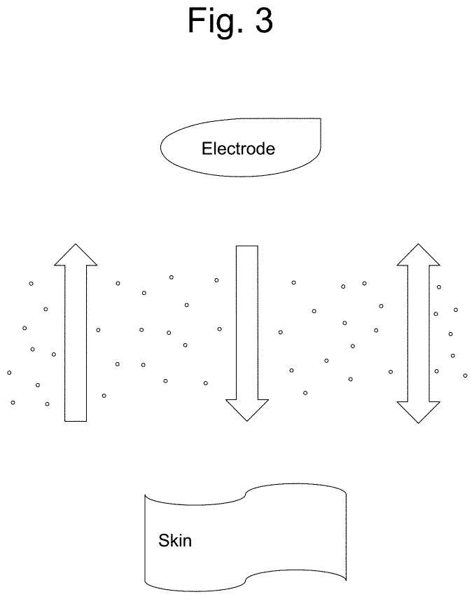

In some embodiments, the systems, devices and methods typically use iontophoresis to control the impedance by controlling electrical current flow between electrodes. As two or more electrodes are needed to measure biosignals, the same electrodes can be used to carry the current produced by the electronics disclosed herein. The embedded electronics of the devices disclosed herein preferably contain switches and current generators that operate as current sources and current sinks to control current flow. Current flow used in the systems and methods disclosed herein can be steady state such as, e.g., direct current (DC), sinusoidal alternating current (AC), pulsed, and/or square wave forms. In preferred embodiments, the current flow protocols can be charge-balanced or not charge-balanced depending on the settings of the system/device. Reverse iontophoresis makes the skin more conductive by moving hydrating/conductive molecules from deep layers of the skin up, and iontophoresis reduces impedance by moving hydrating/conductive molecules from within the electrode down. Both iontophoresis and reverse iontophoresis can be used in the systems and devices disclosed herein if using alternating current (AC) or charge balanced protocols (see, e.g., FIG. 3).

In preferred embodiments, then, this disclosure relates to a self-applied wearable device configured to electrically stimulate a user, generate and/or collect a user's electrophysiological data, comprising one or more electrodes wherein said one or more electrodes are in contact with an outer layer of a skin surface of the user and hydrate the outer layer of the skin via iontophoresis, reverse iontophoresis, and/or a combination thereof to move biological ions and/or polar molecules, such as but not limited to water, from beneath the skin surface into the outer skin surface to increase conductivity and/or to move the molecules from the electrode into the outer skin layer of skin surface to reduced impedance between the outer skin layer and electrode, and wherein the impedance between electrodes is matched and/or minimized. In preferred embodiments, the impedance difference between electrodes is less than about 30 kOhms. In some embodiments, the one or more electrodes use iontophoresis. In some embodiments, the one or more electrodes use iontophoresis and reverse iontophoresis in combination. In some embodiments, the device is configured to measure impedance and actively optimize impedance by iontophoresis, reverse iontophoresis, and/or combination thereof using an optimization algorithm running on a microprocessor of the device or on a computer through a wired or wireless communications path. In some embodiments, the one or more electrodes have the ability to absorb biological ions and/or polar molecules and/or release biological ions and/or polar molecules, wherein the electrode is electrically connected to electronics. In some embodiments, at least some, and in preferred embodiments all of, the electronics of the device are untethered, battery-operated, energy harvesting, and/or wired for power and/or communications. In some embodiments, the device comprises one or more impedance measuring circuits and/or source/sink electrical current generators, wherein said device has the ability to measure electrophysiological data, optionally selected from the group consisting of electroencephalogram (EEG), electrooculography (EOG), electrocardiogram (ECG), electroatriography (EAG), electroventriculography (EVG), intracardiac electrogram (EGM), electrocorticography (ECoG or iEEG), electromyography (EMG), electroretinography (ERG), electronystagmography (ENG), electroolfactography (EOG), electrocochleography (ECOG or ECochG), electrogastrography (EGG), el ectrogastroenterography (EGEG), and electromyography (EMG), and/or other electronic monitoring system. In some embodiments, the device is operated and/or controlled at least in part by at least one algorithm, such as but not limited to those described herein, that rotates electrodes in use and changes polarity of the electrodes to provide impedance conditioning thereby minimizing oxidation of the electrodes. In some embodiments, the device is integrated into a headband, earpiece or wearable sensor net. In some embodiments, the electrical stimulation is transcranial direct current stimulation (tDCS) or electrical muscle stimulation (EMS), but is not limited to such forms of stimulation. In some embodiments, the one or more electrodes comprise a flexible biocompatible conductive polymer. In some embodiments, the one or more electrodes can comprise a hydrogel or a silicone polymer, optionally a polydimethylsiloxane (PDMS) polymer. In some embodiments, the electrodes can further comprise conductive particles selected from silver particles, silver threads, carbon particles, carbon nanotubes, graphene, Ag/AgCl and Ag/AgAl. In some embodiments, this disclosure provides a system comprising at least one of such devices (e.g., a wearable piece, such as a headband, with one or more of such devices associated with and/or embedded within a wearable piece). In some embodiments, the system and/or device is controlled by an impedance optimization algorithm. In some embodiments, this disclosure provides methods for using any such device(s) and/or system(s). Other embodiments are also contemplated herein as would be understood by those of ordinary skill in the art.

The terms “about”, “approximately”, and the like, when preceding a list of numerical values or range, refer to each individual value in the list or range independently as if each individual value in the list or range was immediately preceded by that term. The terms mean that the values to which the same refer are exactly, close to, or similar thereto. Optional or optionally means that the subsequently described event or circumstance can or cannot occur, and that the description includes instances where the event or circumstance occurs and instances where it does not. Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed.

All references cited within this disclosure are hereby incorporated by reference in their entirety. Certain embodiments are further described in the following examples. These embodiments are provided as examples only and are not intended to limit the scope of the claims in any way.

While certain embodiments have been described in terms of the preferred embodiments, it is understood that variations and modifications will occur to those skilled in the art. Therefore, it is intended that the appended claims cover all such equivalent variations that come within the scope of the following claims.

1. A self-applied wearable device configured to electrically stimulate a user, generate and/or collect a user's electrophysiological data, comprising one or more electrodes wherein said one or more electrodes are in contact with an outer layer of a skin surface of the user and hydrate the outer layer of the skin via iontophoresis, reverse iontophoresis, and/or a combination thereof to move biological ions and/or polar molecules from beneath the skin surface into the outer skin surface to increase conductivity and/or to move the molecules from the electrode into the outer skin layer of skin surface to reduced impedance between the outer skin layer and electrode, and wherein the impedance between electrodes is matched and/or minimized.

2. The device of claim 1 wherein the one or more electrodes use iontophoresis or reverse iontophoresis.

3. The device of claim 1 in which one or more electrodes use iontophoresis and reverse iontophoresis in combination.

4. The device of claim 1 that measures impedance and actively optimizes impedance by iontophoresis, reverse iontophoresis, and/or combination thereof using an optimization algorithm running on a microprocessor of the device or on a computer through a wired or wireless communications path.

5. The device of claim 1 comprising one or more electrodes having the ability to absorb biological ions and/or polar molecules and/or release biological ions and/or polar molecules, wherein the electrode is electrically connected to electronics.

6. The device of claim 5 wherein the electronics are untethered, battery-operated, energy harvesting, and/or wired for power and/or communications.

7. The device of claim 1 comprising one or more impedance measuring circuits and/or source/sink electrical current generators, wherein said device has the ability to measure electrophysiological data, optionally selected from the group consisting of electroencephalogram (EEG), electrooculography (EOG), electrocardiogram (ECG), electroatriography (EAG), electroventriculography (EVG), intracardiac electrogram (EGM), electrocorticography (ECoG or iEEG), electromyography (EMG), electroretinography (ERG), electronystagmography (ENG), electroolfactography (EOG), electrocochleography (ECOG or ECochG), electrogastrography (EGG), electrogastroenterography (EGEG), and electromyography (EMG).

8. The device of claim 1 that is operated by an algorithm that rotates electrodes in use and changes polarity of the electrodes to provide impedance conditioning thereby minimizing oxidation of the electrodes.

9. The device of claim 1 wherein the device is integrated into a headband, earpiece, arm band, leg band, wrist band, ring, nosepiece, chest patch or wearable sensor net or patch.

10. The device of claim 1, wherein the electrical stimulation is selected from transcranial direct current stimulation (tDCS) or electrical muscle stimulation (EMS).

11. The device of claim 1, wherein the one or more electrodes comprise a flexible biocompatible conductive polymer.

12. The device of claim 1, wherein the one or more electrodes comprise a hydrogel or a silicone polymer, optionally a polydimethylsiloxane (PDMS) polymer.

13. The device of claim 11, wherein the electrodes further comprise conductive particles selected from silver particles, silver threads, carbon particles, carbon nanotubes, graphene, Ag/AgCl and Ag/AgAl.

14. A system comprising a device of claim 1.

15. The system of claim 14 wherein the device is controlled by an impedance optimization algorithm.

16. A method for using a device of claim 1.
